(19) United States
(12) Patent Application Publication
(10) Pub. No.: US 2025/0205399 A1
(43) Pub. Date: Jun. 26, 2025

(54) PHOTO-REGENERATIVE STENT

(71) Applicant: BOSTON SCIENTIFIC SCIMED, INC., Maple Grove, MN (US)

(72) Inventor: Adam McGraw, Mansfield, MA (US)

(73) Assignee: BOSTON SCIENTIFIC SCIMED, INC., Maple Grove, MN (US)

(21) Appl. No.: 18/988,448

(22) Filed: Dec. 19, 2024

Related U.S. Application Data
(60) Provisional application No. 63/612,829, filed on Dec. 20, 2023.

(51) Int. Cl. A61L 31/18; A61F 2/04; A61L 31/10; A61L 31/16

(52) U.S. Cl. CPC A61L 31/18; A61F 2/04; A61L 31/10; A61L 31/16; A61F 2002/041; A61F 2210/0004; A61F 2210/0076; A61F 2230/0069; A61L 2300/404; A61L 2300/416; A61L 2400/16

(57) ABSTRACT

An illustrative stent includes an elongated tubular member forming a tubular wall, the elongated tubular member configured to move between a radially collapsed configuration and a radially expanded configuration. A polymer coating may be disposed on a surface of the tubular wall. A photolabile layer may be disposed on an inner surface of the polymer coating. The photolabile layer may include a photodegradable polymer.

### CROSS REFERENCE TO RELATED APPLICATIONS

This application claims the benefit of and priority to U.S. Provisional Patent Application Ser. No. 63/612,829, filed on Dec. 20, 2023, the disclosure of which is incorporated herein by reference.

### TECHNICAL FIELD

The present disclosure pertains to medical devices, methods for manufacturing medical devices, and uses thereof. More particularly, the present disclosure pertains to a photo-regenerative stent for implantation in a body lumen, and associated methods.

### BACKGROUND

Implantable stents are devices that are placed in a body lumen, such as the esophageal tract, the gastrointestinal tract (including the intestine, stomach and the colon), tracheobronchial tract, urinary tract, biliary tract, vascular system, etc. to provide support and to maintain the body lumen open. These stents are manufactured by any one of a variety of different manufacturing methods and may be used according to any one of a variety of methods. Of the known stents, delivery systems, and methods, each has certain advantages and disadvantages. For example, some stents may become occluded. Occlusive events in pancreatic and biliary environments can be a combination of local factors, such as, but not limited to inadequate stricture resolution and irregular bile properties that contribute to occlusive biofilm formation and/or related to stent design, such as, but not limited to, coating tackiness and/or an interaction between the wire or sheathing material and bile. Thus, there is an ongoing need to provide alternative stent designs which offer opportunities for prophylactic intervention to restore stent patency responsive to occlusive events or potential occlusive sources without surgical intervention, thereby increasing stent efficacy and/or an operational lifetime of the stent.

### BRIEF SUMMARY

This disclosure provides design, material, manufacturing method, and use alternatives for medical devices. An example medical device may include a stent.

In one example, a stent may comprise an elongated tubular member forming a tubular wall, the elongated tubular member configured to move between a radially collapsed configuration and a radially expanded configuration, a polymer coating disposed on a surface of the tubular wall, and a photolabile layer disposed on an inner surface of the polymer coating, where the photolabile layer includes a photodegradable polymer.

In one example, the polymer coating comprises silicone or polyurethane.

In one example, the polymer coating extends from a proximal end to a distal end of the elongated tubular member, and where the photolabile layer extends from the proximal end to the distal end of the elongated tubular member.

In one example, the polymer coating comprises an opaque filler, a reflective filler, a remotely-stimulated light-emitting element, or any combination thereof.

In one example, the photodegradable polymer is configured to undergo a depolymerization reaction responsive to application of visible light.

In one example, the visible light has a wavelength in a range from about 495 nanometers to about 570 nanometers.

In one example, the photodegradable polymer is configured to undergo a depolymerization reaction responsive to application of ultraviolet light, X-ray radiation, or electromagnetic induction.

In one example, the ultraviolet light has a wavelength in a range from 200 nanometers to 380 nanometers.

In one example, the photolabile layer includes a pharmacological agent.

In one example, the pharmacological agent is an antiproliferative agent.

In one example, the pharmacological agent is an antimicrobial agent.

In one example, the photodegradable polymer further comprises a poly(olefin sulfone), a polythiophene, a polycarbodiimide, a nitrobenzyl group containing polyester, polycarbamate, polyether, polyamide and derivatives and co-polymers thereof.

In one example, the photodegradable polymer comprises about 50 weight percent to 100 weight percent of a total weight of the photolabile layer.

In one example, the photodegradable polymer comprises about 100 weight percent of a total weight of the photolabile layer.

In one example, the photolabile layer comprises a plurality of photolabile layers, the plurality of photolabile layers including a second photolabile layer disposed on an inner surface of the polymer coating, the second photolabile layer configured to undergo depolymerization in response to an applied light having a second wavelength and a first photolabile layer disposed on an inner surface of the second photolabile layer, the first photolabile layer configured to undergo depolymerization in response to an applied light having a second wavelength.

In one example, a system comprises a stent including an elongated tubular member forming a tubular wall, the elongated tubular member configured to move between a radially collapsed configuration and a radially expanded configuration, a polymer coating disposed on a surface of the tubular wall, a photolabile layer disposed on an inner surface of the polymer coating, where the photolabile layer includes a photodegradable polymer and a light-emitting element, where in response to emission of light by the light-emitting element at least a portion of the photolabile layer is configured to liquify and mechanically decouple from a remaining portion of the photolabile layer, the polymer coating, or both.

In one example, a method for restoring patency of a stent implanted in a body of a patient comprises applying light to a photolabile layer of the implanted stent; and where in response to the application of the light, at least a portion of the photolabile layer undergoes depolymerization to form a liquified portion of the photolabile layer.

In one example, the liquified portion of the photolabile layer is configured to mechanically decouple from a remaining portion of the photolabile layer, a polymer coating of the implanted stent, or both.

In one example, the light-emitting element is a remotely-stimulated light-emitting element included in the stent, and wherein the remotely-stimulated light-emitting element is configured to apply the light in vivo of the body of the patient to the photolabile layer responsive to an external and/or transdermal application of X-ray radiation, electromagnetic induction, or both, to the remotely-stimulated light-emitting element.

In one embodiment, the light-emitting element is included in a light-emitting catheter, and wherein the light-emitting element in the light emitting catheter is configured to apply the light in vivo of the body of the patient to the photolabile layer.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a side view of an illustrative endoluminal implant or stent;

FIG. 2A is a schematic cross-sectional view of the occluded endoluminal implant or illustrative stent having photolabile layer prior to light application, taken at line 1-1 of FIG. 1;

FIG. 2B is a schematic cross-sectional view of the illustrative endoluminal implant or stent during light application;

FIG. 2C is a schematic cross-sectional view of the patent illustrative endoluminal implant or stent after light application;

FIG. 3A is a schematic cross-sectional view of another illustrative endoluminal implant or stent having multiple photolabile layers prior to light application, taken at line 1-1 of FIG. 1;

FIG. 3B is a schematic cross-sectional view of the illustrative endoluminal implant or stent after light application at a first wavelength;

FIG. 3C is a schematic cross-sectional view of the illustrative endoluminal implant or stent a period of time following the light application at the first wavelength;

FIG. 3D is a schematic cross-sectional view of the illustrative endoluminal implant or stent after light application at a second wavelength; and

FIG. 4 is a schematic view of an illustrative endoluminal implant or stent deployed within the biliary tree.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

In some instances, it may be desirable to provide an endoluminal implant, or stent, that can deliver luminal patency within the pancreaticobiliary tree of a patient. The relatively narrow biliary tract ducts consist of a series of bifurcations linking the liver, gallbladder, and pancreas via the papilla to the duodenal space for the transportation of bile and related enzymic substances for many metabolic functions but most commonly the body's ability to digest and absorb fats and vitamins D and K. However, blockages may occur in the pancreaticobiliary tree due to tumor related duct narrowing, stricture formation, infections, or stone and sludge generation among other etiologies. Endoscopic retrograde cholangiopancreatography (ERCP) is used to diagnose and treat these duct narrowings, regardless of the malignant or benign nature of the disease. Commonly a fully covered self-expanding metal stent (SEMS) may be used such that the radial forces of the stent scaffold the stricture. However, occlusions may occur within the stent after stent placement. Occlusive events can be a combination of local factors such as, but not limited to, inadequate stricture resolution and/or irregular bile properties. Occlusion can also be related to stent design components such as, but not limited to, coating tackiness and/or an interaction between the wire of the stent and bile (both causing drag and pooling of bile which accumulates over a time course). Due to the remote nature of a placed SEMS, the ability to diagnose and interact with a potential occlusive event is limited and typically can only be resolved through intervention at a stage when the occlusive event has demonstrated an exacerbation of a patient's symptoms (e.g., jaundice, abdominal pain, etc.). Occlusion may result in increased procedural charges due to reintervention and stent replacement, patient discomfort even though the stent device itself may be functioning effectively as a scaffold, retarding of the vessel, and/or an unjustified reputation for particular device families as being less efficacious or brands spurned due to historical or anecdotal knowledge of occlusive or suggestive occlusive events. The present disclosure is directed towards alternative stent designs which offer opportunities for prophylactic or preventative intervention to restore the patency of stents without surgical intervention thereby increasing stent efficacy and/or extending an operational lifetime of the stent. While the present disclosure is described with respect to the pancreaticobiliary ductal system, the devices, systems, and/or methods described herein may be used in stents or endoluminal implants positioned in other parts of the body, such as, but not limited to, bodily tissue, bodily organs, vascular lumens, non-vascular lumens and combinations thereof, such as, but not limited to, in the coronary or peripheral vasculature, trachea, bronchi, colon, small intestine, esophagus, biliary tract, urinary tract, prostate, brain, stomach, and the like.

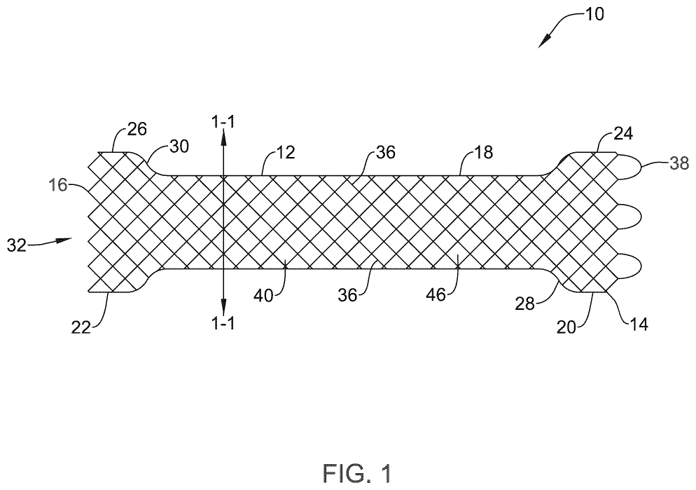

FIG. 1 illustrates a side view of an illustrative endoluminal implant 10, such as, but not limited to, a stent. In some instances, the stent 10 may be formed from an elongated tubular member 12. While the stent 10 is described as generally tubular, it is contemplated that the stent 10 may take any cross-sectional shape desired. The stent 10 may have a first, or proximal end 14, a second, or distal end 16, and an intermediate region 18 disposed between the first end 14 and the second end 16. The stent 10 may include a lumen 32 extending from a first opening adjacent the first end 14 to a second opening adjacent to the second end 16 to allow for the passage of food, fluids, etc.

The stent 10 may be expandable from a first radially collapsed configuration (not explicitly shown) to a second radially expanded configuration. In some cases, the stent 10 may be deployed to a configuration between the collapsed configuration and a fully expanded configuration. The stent 10 may be structured to extend across a stricture and to apply a radially outward pressure to the stricture in a lumen to open the lumen and allow for the passage of substances.

In some embodiments, the proximal end 14 of the stent 10 may include a plurality of loops 38. The loops 38 may be configured to receive a retrieval tether or suture (not explicitly shown) interwoven therethrough, or otherwise passing through one or more of the loops 38. The retrieval suture may be used to collapse and retrieve the stent 10, if so desired. For example, the retrieval suture may be pulled like a drawstring to radially collapse the proximal end 14 of the stent 10 to facilitate removal of the stent 10 from a body lumen.

The stent 10 may have a woven structure, fabricated from a number of filaments or struts 36 forming a tubular wall. In some embodiments, the stent 10 may be knitted or braided with a single filament or strut interwoven with itself and defining open cells 46 extending through the thickness of the tubular wall of the stent 10. In other embodiments, the stent 10 may be braided with several filaments or struts interwoven together and defining open cells 46 extending along a length and around the circumference of the tubular wall of the stent 10. The open cells 46 may each define an opening from an outer surface of the tubular wall to an inner surface of the tubular wall (e.g., through a thickness thereof) that is free from the filaments or struts 36. Some exemplary stents including braided filaments include the WallFlex®, WALLSTENT®, and Polyflex® stents, made and distributed by Boston Scientific, Corporation. In another embodiment, the stent 10 may be knitted, such as the Ultraflex™ stents made by Boston Scientific, Corporation. In yet another embodiment, the stent 10 may be of a knotted type, such as the Precision Colonic™ stents made by Boston Scientific, Corporation. In still another embodiment, the stent 10 may be a laser cut tubular member, such as the EPIC™ stents made by Boston Scientific, Corporation. A laser cut tubular member may have an open and/or closed cell geometry including one or more interconnected monolithic filaments or struts defining open cells 46 therebetween, with the open cells 46 extending along a length and around the circumference of the tubular wall. The open cells 46 may each define an opening from an outer surface of the tubular wall to an inner surface of the tubular wall (e.g., through a thickness thereof) that is free from the interconnected monolithic filaments or struts. In some instances, an inner and/or outer surface of the tubular wall of the stent 10 may be entirely, substantially, or partially, covered with a polymer coating 40, as will be described in more detail herein. The polymer coating 40 may extend across and/or occlude one or more, or a plurality of the cells 46 defined by the filaments or struts 36. In some cases, the stent 10 may be a self-expanding stent (SES), although this is not required.

In some instances, in the radially expanded configuration, the stent 10 may include a first end region 20 proximate the proximal end 14 and a second end region 22 proximate the second end 16. In some embodiments, the first end region 20 and the second end region 22 may include retention features or anti-migration flared regions 24, 26 having enlarged diameters relative to the intermediate region 18. The anti-migration flared regions 24, 26, which may be positioned adjacent to the first end 14 and the second end 16 of the stent 10, may be configured to engage an interior portion of the walls of the body lumen. In some embodiments, the retention features, or flared regions 24, 26 may have a larger diameter than the cylindrical intermediate region 18 of the stent 10 to prevent the stent 10 from migrating once placed in the body lumen. It is contemplated that the transition 28, 30 from the cross-sectional area of the intermediate region 18 to the retention features or flared regions 24, 26 may be gradual, sloped, or occur in an abrupt step-wise manner, as desired.

In some embodiments, the first anti-migration flared region 24 may have a first outer diameter and the second anti-migration flared region 26 may have a second outer diameter. In some instances, the first and second outer diameters may be approximately the same, while in other instances, the first and second outer diameters may be different. In some embodiments, the stent 10 may include only one or none of the anti-migration flared regions 24, 26. For example, the first end region 20 may include an anti-migration flare 24 while the second end region 22 may have an outer diameter similar to the intermediate region 18. It is further contemplated that the second end region 22 may include an anti-migration flare 26 while the first end region 20 may have an outer diameter similar to an outer diameter of the intermediate region 18. In some embodiments, the stent 10 may have a uniform outer diameter from the first end 14 to the second end 16. It is contemplated that the outer diameter of the stent 10 may be varied to suit the desired application.

It is contemplated that the elongated tubular member of the stent 10 can be made from a number of different materials such as, but not limited to, metals, metal alloys, shape memory alloys, and/or polymers, as desired, enabling the stent 10 to be expanded into shape when accurately positioned within the body. In some instances, the material may be selected to enable the stent 10 to be removed with relative ease as well. For example, the elongated tubular member of the stent 10 can be formed from alloys such as, but not limited to, nitinol and Elgiloy®. Depending on the material selected for construction, the stent 10 may be self-expanding or require an external force to expand the stent 10. In some embodiments, composite filaments may be used to make the stent 10, which may include, for example, an outer shell or cladding made of nitinol and a core formed of platinum or other radiopaque material. It is further contemplated the elongated tubular member of the stent 10 may be formed from polymers including, but not limited to, polyethylene terephthalate (PET). In some instances, the filaments of the stent 10, or portions thereof, may be bioabsorbable or biodegradable, while in other instances the filaments of the stent 10, or portions thereof, may be biostable.

As described above, the inner and/or outer surface of the tubular wall of the stent 10 may be entirely, substantially, or partially covered with a polymer coating 40. The polymer coating 40 may be silicone, polyurethane, or other flexible polymeric material. The polymer coating 40 may be generally taut and extend in the same plane as the struts 36. The coating can be applied as described herein.

The polymer coating 40 may span or be disposed within openings or interstices 46 defined between adjacent stent filaments or struts 36 of the scaffold structure. For example, the polymer coating may extend into the openings 46 defined between adjacent stent struts 36 and form an interface region. Further, the polymer coating 40 may additionally extend between adjacent filaments or struts 36, thereby filling any space between adjacent filaments or strut members 36, and thus prevent tissue ingrowth into the lumen of the stent 10.

The stent 10 can include a photodegradable polymer (not shown in FIG. 1) in one or more photolabile layers (not shown in FIG. 1) on at least a portion of the stent 10, as described herein. In some examples, the one or more photolabile layers may be arranged to suit a particular condition under treatment or the perceived location of potential occlusion. For example, if an occlusion is predicted to potentially occur higher in the biliary duct 100 (e.g., potentially due to stones, debris, etc.), the one or more photolabile layers may be located at least at the proximal end 14 of the stent 10. In another example, if an occlusion is predicted to potentially occur at the lower aspect of the stent 10 (e.g., potentially due to food impaction, etc.), the one or more photolabile layers may be located closer to the distal end 16 of the stent 10 (e.g., near the duodenal end).

The one or more photolabile layers may substantially cover a portion of the stent. For instance, one or more photolabile layers extend over a length and/or circumference of the stent 10. The photodegradable polymer may be uniformly dispersed within the one or more photolabile layers and thus may promote the uniform depolymerization and liquification and/or sublimation of the one or more photolabile layers. As detailed herein, the photodegradable polymer and thus the one or more photolabile layers may be configured to undergo depolymerization (e.g., undergo a depolymerization chain reaction) into the constituent components of the photodegradable polymer responsive to exposure of the photodegradable polymer to in vivo light (e.g., visible light and/or ultraviolet light).

For example, the photodegradable polymer may be a poly(olefin) sulfone that may undergo depolymerization responsive to application of in vivo light such that the original olefin monomer and the sulfur dioxide are regenerated from a primary chain of the poly(olefin sulfone), among other possible types of photodegradable polymers. As detailed herein, the photo-induced depolymerization of photodegradable polymer forming the one or more photolabile layers may intrinsically cause the one or more photolabile layers to mechanically decouple from a remaining portion of the one or more photolabile layer, a polymer coating of the implanted stent, or both. The mechanical decoupling of one or more photolabile layers may in turn release any biliary sludge coupled to the one or more photolabile layers and thereby restore the patency of a stent, as detailed herein. That is, in contrast to other approaches (e.g., those which employ stents that require direct mechanical removal (e.g., invasive surgery to remove an occluded stent), the approaches herein can restore stent patency in the absence of a direct mechanical intervention due at least in part to the particular combination of materials employed herein (e.g., the photolabile layer). For instance, the photolabile layer can be selectively liquified responsive to the application of light, as described herein. In some embodiments, the photolabile layer may be configured to remain stable (e.g., not undergo depolymerization) prior to, during, and/or following implantation of the stent 10 into a patient. For example, in some embodiments a photolabile layer in the stent 10 may be configured to undergo a depolymerization reaction only when the stent 10 is at a physiological temperature and is exposed to light (e.g., in vivo light). For instance, prior to implantation and being at a physiological temperature the stent 10 may not undergo a depolymerization reaction, even if exposed to light prior to or during implantation of the stent 10 into the patient.

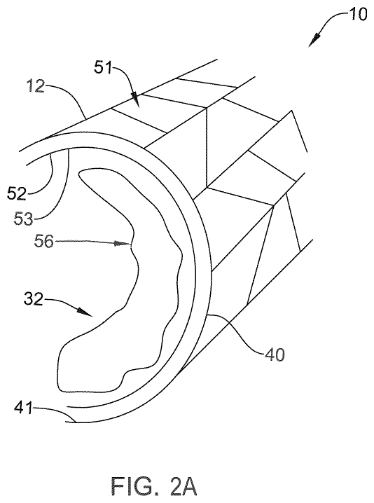

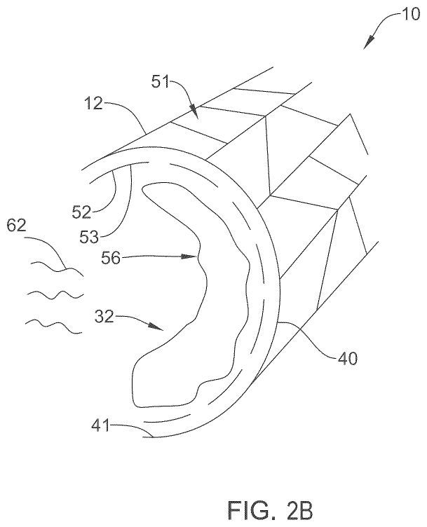

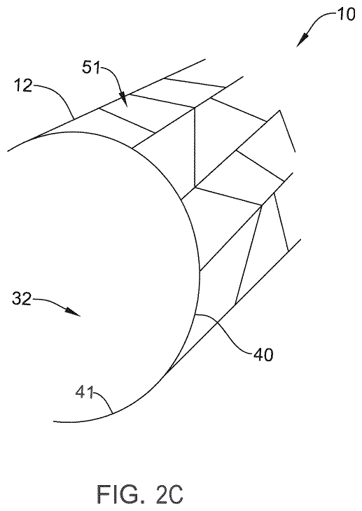

FIG. 2A is a schematic cross-sectional view of the occluded endoluminal implant or illustrative stent 10 having a photolabile layer prior to light application, taken at line 1-1 of FIG. 1. FIG. 2B is a schematic cross-sectional view of the illustrative endoluminal implant or stent 10 during light application. FIG. 2C is a schematic cross-sectional view of the illustrative endoluminal implant or stent 10 showing patency after light application.

As illustrated in FIGS. 2A-2C, the stent 10 can include the elongated tubular member 12 forming a tubular wall 51. The tubular wall 51 can extend along at least a portion of the length of the stent 10 between a distal end to a proximal end of the stent 10. For instance, the tubular wall 51 can have a length that is at least equal to a cylindrical intermediate region (e.g., the cylindrical intermediate region 18, as illustrated in FIG. 1) of the stent 10. In some embodiments, the tubular wall 51 can extend an entire length of the stent 10 between a distal end and a proximal end of the stent 10.

The stent 10 can include a polymer coating 40 disposed on a surface of the tubular wall 51. For instance, the polymer coating 40 can be disposed at least on an inner surface of the tubular wall 51. The polymer coating 40 can be disposed along at least a portion of the length and at least a portion of a circumference of the tubular wall 51. In some embodiments, the polymer coating 40 can extend from a proximal end to a distal end of the elongated tubular member 12. For instance, the polymer coating 40 can be disposed along an entire length and around an entire circumference of the tubular wall 51, where the tubular wall 51 extends at least a portion of or an entire length between a distal end and a proximal end of the stent 10. In some embodiments, the polymer coating 40 includes silicone or polyurethane. For instance, the polymer coating 40 may be a clear silicone or clear silicon-based coating, among other possibilities.

In some embodiments, the polymer coating 40 may include an opaque filler, a reflective filler, a remotely-stimulated light-emitting element (e.g., a transdermally-stimulated light-emitting element), or any combination thereof. For instance, in some embodiments, the polymer coating 40 comprises an opaque filler, a reflective filler, or both. For instance, in some embodiments, the polymer coating 40 includes an opaque filler such as titanium dioxide, zinc oxide, other transition metal oxides, carbon nanotubes, gas-expanded polyetheylenes, among other possible opaque fillers. In some embodiments, the polymer coating 40 includes a reflective filler such as filaments or microparticles of oxidation state zero aluminum, gold, stainless steel or other metals, cholesteric liquid crystals, quantum dots, phosphorescent agents, 1-dimensional photonic crystals, silicon dioxide microspheres, among other possible reflective fillers. Inclusion of the opaque filler, the reflective filler, or both in the polymer coating 40 may increase a percentage of the applied light, as described herein, that is received by the photodegradable polymer in the photolabile layer. For instance, the presence of the opaque filler and/or filler in the polymer coating 40 may redirect (e.g., reflect) applied light that might otherwise be transmitted through the polymer coating 40 (e.g., a clear silicon polymer coating) to instead cause the light to be redirected to the photodegradable polymer in a photolabile layer. Thus, the photolabile layer may readily undergo complete depolymerization and/or a time associated with the in vivo application of light may be reduced. In some embodiments, a weight of the filler (e.g., the opaque filler and/or the reflective filler) can be in a range from about 1 to about 50 weight percent of a total weight of the polymer coating 40. All individual values and sub-ranges from about 1 weight percent to about 50 weight percent are included. For instance, the filler may be present in a range from about 1 to about 10 weight percent, from about 5 to about 10 weight percent, from about 1 to about 20 weight percent, or from about 10 to about 20 weight percent of the total weight of the polymer coating 40, among other possibilities.

The stent 10 can include a photolabile layer 52 disposed at least on an inner surface 41 of the polymer coating 40. For instance, the photolabile layer 52 can be disposed only on an inner surface 41 of the polymer coating 40. In some embodiments, the photolabile layer 52 can be disposed directly on (without intervening layers or elements) the inner surface 41 of the polymer coating 40. The photolabile layer 52 can be disposed along at least a portion of the length and along at least a portion of a circumference of the polymer coating 40. In some embodiments, the photolabile layer 52 can extend from the proximal end to the distal end of the elongated tubular member. In some embodiments, the photolabile layer 52 can be disposed along an entire length and around an entire circumference of the polymer coating 40.

In some embodiments, the photolabile layer 52 can include a poly(olefin sulfone), a nitrobenzyl group containing polymer, such as polyesters, polycarbamates, polycarbonates, and polyamides, among others. In some cases, the photolabile layer 52 can be formed of a bio-degradable, bio-inert, and/or non-toxic photo-responsive polymer.

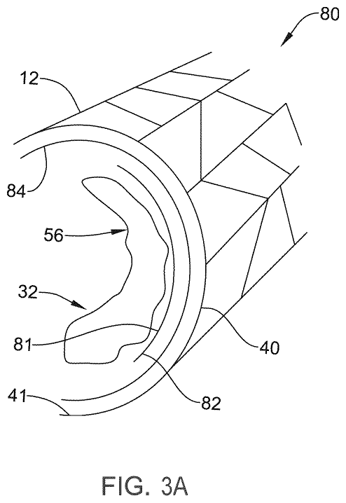

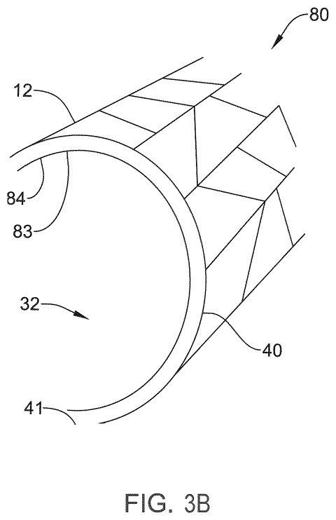

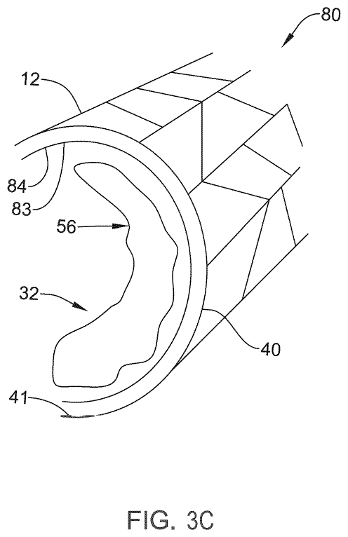

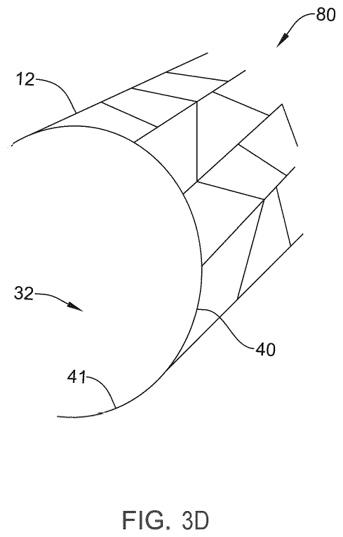

In some embodiments, the photolabile layer 52 can be an individual photolabile layer such as the photolabile layer 52, as illustrated in FIG. 2A. However, in some embodiments the photolabile layer can include a plurality of photolabile layers (e.g., having differing amounts and/or different types of photodegradable polymers included therein), as described herein with respect to FIGS. 3A-3D.

As illustrated in FIG. 2A, a lumen 32 of the stent 10 can become at least partially occluded by material such as biliary sludge 56. For instance, over time the biliary sludge 56 may adhere to an inner surface 53 of the photolabile layer 52 of the stent 10. The biliary sludge 56 may extend radially from the inner surface 53 of the photolabile layer 52 into the lumen 32, thereby blocking at least a portion of a flow through the lumen 32 and thus may inhibit efficacy of the stent 10.

As mentioned, the photolabile layer 52 can include a photodegradable polymer. At least a portion of the photolabile layer 52 can be configured to undergo a depolymerization reaction, liquify, and mechanically decouple from a remaining portion of the photolabile layer 52, the polymer coating 40, or both. For instance, the entire photolabile layer 52 (e.g., along the entire circumference of the inner surface 41 of the polymer coating 40) can be configured to undergo a depolymerization reaction, liquify and mechanically decoupled from the inner surface 41 of the polymer coating 40 in response to application of light (e.g., in vivo light that is applied internal to a patient's body).

As illustrated in FIG. 2B, the application of the in vivo light (represented as element 62) can cause the depolymerization, liquification, and mechanical decoupling of at least a portion of the photolabile layer 52 from the inner surface 41 of the polymer coating 40. Application of the in vivo light can continue until some or all of the photolabile layer 52 undergoes a depolymerization reaction, is liquified (as a result of undergoing the depolymerization reaction), and mechanically decouples from the inner surface 41 of the polymer coating 40.

For instance, as illustrated in FIG. 2C the entire photolabile layer 52 can be liquified and mechanically decoupled from the inner surface 41 of the polymer coating 40. The selective depolymerization, liquefaction, and mechanically decoupling of at least a portion of the photolabile layer 52 responsive to the application of an in vivo light can restore the patency of the stent 10. Namely, the biliary sludge 56 that was previously coupled to at least a portion of the inner surface 53 of the photolabile layer 52 along with the liquified photolabile layer 52 corresponding to the at least the portion of the inner surface 53 to which the biliary sludge 56 was adhered can be dislodged from the stent 10. Thus, a diameter of the lumen 32 can be restored to substantially biliary sludge-free diameter without surgical intervention, thereby increasing stent efficacy and/or an operational lifetime of the stent. The polymer coating 40 can remain intact during and following the selective liquification of the photolabile layer 52. For instance, the entire polymer coating layer 40 can remain intact during and following the selective liquification of the photolabile layer 52. The presence of the intact polymer layer 40 can promote aspects herein, such as mitigating tissue in-growth into the lumen 32.

FIG. 3A is a schematic cross-sectional view of another illustrative endoluminal implant or stent 80 having a plurality of photolabile layers prior to exposure to in vivo light, taken at line 1-1 of FIG. 1. FIG. 3B is a schematic cross-sectional view of the illustrative endoluminal implant or stent 80 after light application at a first wavelength. FIG. 3C is a schematic cross-sectional view of the illustrative endoluminal implant or stent 80 a period of time following the light application at the first wavelength. FIG. 3D is a schematic cross-sectional view of the illustrative endoluminal implant or stent 80 after light application at a second wavelength. The stent 80 illustrated in FIGS. 3A-3D is analogous to the stent 10 illustrated in FIG. 2A-2C, with the difference that the stent 80 includes a plurality of photolabile layers rather than an individual photolabile layer.

The stent 80 can include the elongated tubular member 12 forming a tubular wall 51 and a polymer coating 40 disposed on at least on an inner surface of the tubular wall 51. The stent 80 includes a plurality of photolabile layers. For instance, the stent can include two photolabile layers such as a first photolabile layer 82 and a second photolabile layer 84. The second photolabile layer 84 can be disposed on an inner surface 41 of the polymer coating 40 and a first photolabile layer 82 can be disposed on an inner surface 83 of the second photolabile layer 84. As illustrated in FIG. 3A, the second photolabile layer 84 can be disposed directly on (without intervening layers or elements) an inner surface 41 of the polymer coating 40 and the first photolabile layer 82 can be disposed directly on an inner surface 83 of the second photolabile layer 84. In some embodiments, the second photolabile layer 84 can be disposed directly on (without intervening layers or elements) and extend around an entire circumference of the inner surface 41 of the polymer coating 40 and the first photolabile layer 82 can be disposed directly on and extend around an entire circumference of the inner surface 83 of the second photolabile layer 84.

The second photolabile layer 84 (e.g., an inner photolabile layer) can be configured to undergo a depolymerization reaction in response to applied light having a second wavelength. The first photolabile layer 82 (e.g., an outer photolabile layer) can be configured to undergo a depolymerization reaction in response to an applied light having a first wavelength. The first photolabile layer 82 and the second photolabile layer 84 can be configured to undergo depolymerization reactions and thus liquify in response to different wavelengths of light. For instance, the second photolabile layer 84 can be configured to undergo a depolymerization reaction at a wavelength that is higher or lower than a light wavelength at which the first photolabile layer 82 is configured to undergo a depolymerization reaction. Thus, as described herein, the first photolabile layer 82 can be liquified and mechanically decoupled from the second photolabile layer 84 which can remain intact during the application of light to the stent and liquification of the first photolabile layer 82. In this way, the stent 80 can undergo a plurality of applications of light to iteratively liquify some of the plurality of photolabile layers, thereby permitting restoring patency of the stent 80 a plurality of times. For instance, the stent 80 can undergo the application of light at in two different time periods (e.g., initially to liquify and remove the first photolabile layer 82 and subsequently to liquify and remove the second photolabile layer 84), and thereby restore the patency of the stent 80 on two separate occasions to increase stent efficacy and operation lifetime of the stent.

The first photolabile layer 82 and the second photolabile layer 84 may be formed as separate layers. The first photolabile layer 82 and the second photolabile layer 84 may be formed from the same material or different materials, as desired. In some embodiments, the first photolabile layer 82 and the second photolabile layer 84 can include different concentrations and/or different types of photodegradable polymers. For instance, the second photolabile layer 84 can include a second type and/or concentration of a photodegradable polymer such that the second photolabile layer 84 is configured to undergo depolymerization responsive to application of light at a second wavelength (e.g., responsive to application of ultraviolet light, X-ray radiation, and/or electromagnetic induction), while the first photolabile layer 82 can include a first type and/or concentration of a photodegradable polymer such that the first photolabile layer 82 is configured to undergo a depolymerization reaction in responsive to light at a first wavelength (e.g., visible light) that is different than the second wavelength. In such embodiments, the second photolabile layer 84 may be configured to undergo a depolymerization reaction responsive to the application of light at a second wavelength which is higher or lower than a first wavelength at which the first photolabile layer 84 is configured to undergo a depolymerization reaction.

In some embodiments, the light may be selectively applied along the length of the stent 80. In FIG. 3A, the stent 80 is in an initial state with no in vivo light being applied. As can be seen, the first photolabile layer 82 and the second photolabile layer 84 are each present in the stent 80. In the presence of the applied light at a first wavelength, the photodegradable polymer in the first photolabile layer 82 may undergo a depolymerization reaction such that the first photolabile layer 82 is liquified and mechanically detaches from at least a portion of an inner surface 83 of the second photolabile layer 84. As such, the biliary sludge 56 (illustrated in FIG. 3A as being attached to an inner surface 81 of the first photolabile layer 82) can be removed from the lumen 32 of the stent 80. Thus, as illustrated in FIG. 3B, the first photolabile layer 82 and the biliary sludge 56 are no longer present in the stent 80 following light application at the first wavelength.

Subsequent to the depolymerization, liquification, and mechanical removal of the first photolabile layer 82 and the biliary sludge 56 initially present in the stent 80, additional biliary sludge may accumulate on an inner surface 83 of the second photolabile layer 84, as the inner surface 83 is now being exposed to and in fluidic contact with material in the lumen 32. As illustrated in FIG. 3C, biliary sludge 56 has accumulated on the inner surface 83 of the second photolabile layer 84 after a period of time following the application of in vivo light at the first wavelength. In FIG. 3C, the stent 80 is in another initial state without the application of in vivo light.

In the presence of the applied light at a second wavelength, the photodegradable polymer in the second photolabile layer 84 may undergo a depolymerization (e.g., that is the same or different than the depolymerization reaction previously undergone by the first photolabile layer 82) such that the second photolabile layer 84 is liquified and mechanically detaches from at least a portion of the inner surface 41 of the polymer coating 40. As such, the biliary sludge 56 (illustrated in FIG. 3C) can be removed from the lumen 32 of the stent 80. Thus, as illustrated in FIG. 3D, the first photolabile layer 82, the second photolabile layer 84, and the biliary sludge 56 (e.g., as illustrated in FIG. 3A and as illustrated in FIG. 3C) are no longer present in the stent 80 following light application at the second wavelength. In some cases, the first wavelength may be a lower wavelength than the second wavelength. However, in some instances, the first wavelength can be equal to or greater than the second wavelength. In some cases, the first wavelength and the second wavelength can be respective wavelengths within a range from about 200 nanometers (nm) to about 900 nm. For instance, the first wavelength can correspond to visible light having a wavelength in a range from about 400 nm to about 800 nm or from about 400 nm to about 700 nm, and the second wavelength can correspond to ultraviolet light having a wavelength corresponding to about 200 nm to about 400 nm from 200 nm to 380 nm, among other possibilities. In some embodiments, a photolabile layer such as the first photolabile layer 82 and/or the second photolabile layer 84 may be configured to undergo depolymerization responsive to application of “green” visible light having a wavelength in a range from about 495 nm to about 570 nm.

In some embodiments, the stents herein can include a pharmacological agent. Examples of suitable pharmacological agents include antiproliferative agents and antimicrobial agents. For instance, a pharmacological agent may be included in a photolabile layer such as the photolabile layer 52. Inclusion of the pharmacological agent in the photolabile layer 52 can promote aspects herein such as providing a bulk or bolus release of the pharmacological agent when the photolabile layer 52 undergoes a depolymerization reaction and liquification responsive to the application of in vivo light to the photolabile layer 52. For instance, an antiproliferative agent and/or an antimicrobial agent may be released to mitigate tissue ingrowth into the stent following the liquification and removal of the photolabile layer 52. It is contemplated that the liquefaction and removal of the photolabile layer 52 may render the stent prone to tissue in growth and/or microbial growth on a newly exposed surface (e.g., a newly exposed photolabile layer or a newly exposed polymer coating). Thus, the bolus release of the antiproliferative agents and/or antimicrobial agents included in the photolabile layer 52 at the same time as the liquefaction of the photolabile layer 52 may mitigate any tissue growth into and/or bacterial adhesion on to the de novo luminal stent surface. In embodiments with a plurality of photolabile layers it is contemplated that at least some of the plurality of photolabile layers may include a pharmacological agent. For instance, in some embodiments, each of the photolabile layers may include a pharmacological agent, among other possibilities. In some embodiments, a weight of the pharmacological agent can be in a range from about 1 to about 10.0 weight percent of a total weight of the photolabile layer. All individual values and sub-ranges from about 1 weight percent to about 10 weight percent are included. For instance, the pharmacological agent may be present in a range from about 1 to about 10 weight percent, from about 1 to about 5 weight percent, from about 1 to about 3 weight percent, or from about 1 to about 2 weight percent of a total weight of the photolabile layer (e.g., the first and/or second photolabile layer), among other possibilities.

In some embodiments, a combined weight of the photodegradable polymer and a pharmacological agent can be in a range from about 70 to 100 weight percent, from about 80 to 100 weight percent, or from about 90 to 100 of a total weight of the polymer coating 40. For instance, fillers or other additives may make up a remainder of the total weight of the polymer coating 40. However, in some embodiments a combined weight of the photodegradable polymer and a pharmacological agent can be 100 weight percent of a total weight of the polymer coating 40.

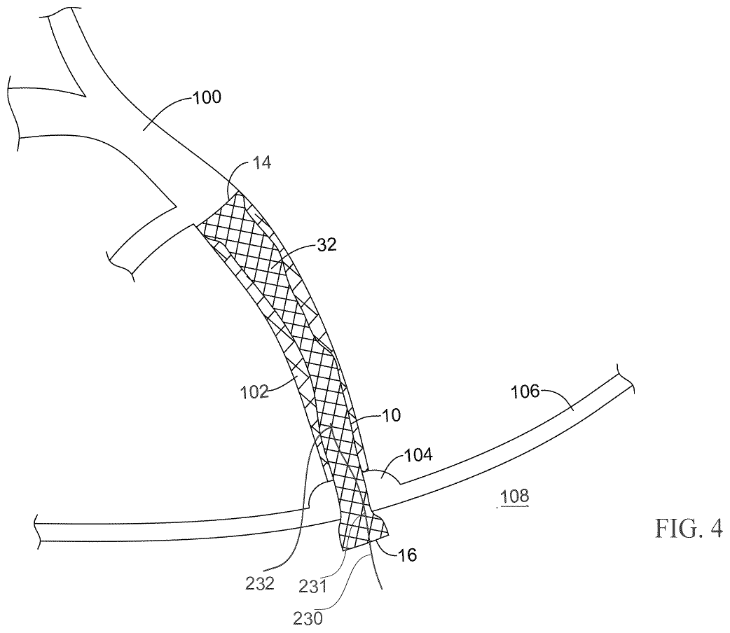

FIG. 4 depicts a schematic view of the illustrative stent 10 deployed within the biliary tree. In the illustrated example, the stent 10 is positioned within a biliary duct 100 and is positioned across a stricture 102. In some embodiments, an entirety of a stent may be exposed to the in vivo light at a same time. In yet other embodiments, the in vivo light may be applied to a region of the stent adjacent to the stricture 102 or another diseased site.

The distal end 16 of the stent 10 extends past the papillary mass 104, through the duodenal wall 106 and into the duodenum 108. The stent 10 may be positioned within the biliary tree via endoscopic retrograde cholangiopancreatography (ERCP). For example, the stent 10 may be guided to the biliary location and subsequently deployed, as shown in FIG. 4. The patient may be discharged with the stent 10 in situ. For subsequent maintenance of the stent 10 (e.g., to restore patency of the stent 10 by resolving occlusion of the lumen 32 of the stent) after a period of time, the patient may be guided through a minimally- or non-invasive treatment option. For example, the stent 10 may be subjected to a light to cause the photodegradable polymer in a photolabile layer of the stent 10 to undergo a depolymerization reaction. In one embodiment, the light emitting element 232 may include light fibers or light-emitting diodes affixed to small-diameter catheters 230 that are deployed to the occluded lumen using standard endoscopic procedures that do not require removal or transposing of the placed stent 10. In an additional embodiment, the stent 10 may include a remotely-stimulated light emitting element, such as a transdermally-stimulated light emitting element. The remotely-stimulated light emitting element may be small light fibers or LEDs that are directly affixed to the stent 10 (e.g., disposed on or in the polymer layer 40 and/or disposed on the struts 36) prior to deployment and are energized by transdermal electromagnetic induction to achieve wireless electrical transfer (e.g., electro-motive force), avoiding the need for direct biliary access e.g., via an elongate light-emitting catheter. In another embodiment, the stent may include a remotely-stimulated light emitting element in the form of reagents (such as fluorescein, aluminum phthalocyanine, proflavin, among others) that undergo X-ray-induced luminescence (XML) that are incorporated into the polymer coating 40 of the stent 10. Administration of electromagnetic radiation and/or low energy X-rays to the treatment area (e.g., transdermal administration of electromagnetic radiation and/or low energy X-rays to the treatment area) will induce light production within the stent 10 and thereby degrade the photolabile layer without requiring direct stent access e.g., via an elongate light-emitting catheter.

In some embodiments, the photodegradable polymer and therefore the photolabile layer is configured to undergo a depolymerization reaction at a temperature that is equal to a physiological temperature (e.g., 37 degrees Celsius). Thus, the photolabile layer can be selectively liquified responsive to the application of in vivo light, and yet may not subject surrounding tissue to elevated temperatures. Additionally, the photolabile layer may remain stable (e.g., does not undergo a depolymerization reaction) prior to or during implant of the stent including the photolabile layer, even if the stent is exposed to light prior to or during implantation.

In some embodiments, the photodegradable polymer can be included in a plurality of photolabile layers that are each configured to undergo depolymerization responsive to the application of in vivo light at different wavelengths. For instance, a first photolabile layer can include a type and/or amount of photodegradable polymer such that the first photolabile layer is configured to undergo a depolymerization reaction (e.g., such that the first photolabile layer undergoes liquification) responsive to a first wavelength of light and a second photolabile layer can include a different a type and/or amount of photodegradable polymer such that the second photolabile layer is configured to undergo a depolymerization responsive to a second wavelength of light. In some cases, the first wavelength of light can be different than the second wavelength by a threshold amount (e.g., 50, 100 nm, etc.). In some embodiments, the first photolabile layer may be configured to undergo a depolymerization reaction responsive to an in vivo application of a certain color of visible light (e.g., yellow light) and the second photolabile layer may be configured to undergo a depolymerization reaction responsive to an in vivo application of a different color of visible light (e.g., green or blue light).

As mentioned, at least a portion of the photolabile layer can be configured to undergo a depolymerization reaction, liquify (e.g., change from a solid state to a liquid state) in response to the application of in vivo light. The liquified portion of the photolabile layer can be configured to mechanically decouple from a surface of a remaining portion of the photolabile layer, a surface of a polymer coating on the implanted stent, or both. Thus, any biliary sludge coupled to the liquified portion of the photolabile layer can be released from the stent. The photolabile layer can be configured to remain liquified (e.g., responsive to the cessation of the application of the in vivo light) once exposed to the in vivo light and having undergone depolymerization. For instance, the photolabile layer can be configured to remain liquified while inside of a patient's body (e.g., while at or above a physiological temperature) so the liquified photolabile layer, along with any released biliary sludge, can readily pass through and out of the patient's body. The selective depolymerization and liquification of at least a portion of the photolabile layer can thereby restore patency of the lumen 32 of the stent 10. It is contemplated that the time period between applications of the in vivo light may vary depending on whether or not the patient is symptomatic (e.g., showing signs of an occlusion), among other factors. In some cases, the time period between implant of the stent and application of the in vivo light may be in the range of weeks, months, or years. In some cases, the time period between a first application of the in vivo light (e.g., at a first wavelength) and a second application of the in vivo light (e.g., at a second wavelength that is different than the first wavelength) may be in the range of weeks, months, or years. It is further contemplated that the in vivo light may be applied at an outpatient facility, at a step-down facility, or as a home care option with the correct instructions. The application of in vivo light via a light-emitting element of a light-emitting catheter to the stent 10 may be a less invasive and less costly intervention compared to full surgical correction of an occlusion.

For instance, the in vivo light may be applied internally via an elongate light-emitting catheter 230 and therefore is a less invasive and less costly intervention compared to full surgical correction of an occlusion. The elongate light-emitting catheter 230 may comprise an elongate catheter shaft 231 that is inserted into a patient. For instance, as depicted in FIG. 4 the elongate catheter shaft 231 may be extend into an artery of the patient adjacent to the stent 10. Thus, light can be applied in vivo to the inner surface of the stent 10, as described herein. As mentioned, the application of in vivo light via a light-emitting element of a light-emitting catheter to the stent 10 may be a less invasive and less costly intervention compared to full surgical correction of an occlusion. For instance, the in vivo light may be applied internally via a light-emitting catheter 231 and therefore is a less invasive and less costly intervention compared to full surgical correction of an occlusion. The stent 10 and the elongate light-emitting catheter 230 can be positioned interior to the patient adjacent to the desired treatment region. For instance, the stent 10 and the elongate light-emitting catheter 230 can be positioned adjacent to the biliary tree. It is contemplated that the stent 10 and the elongate light-emitting catheter 230 may be positioned at any location within the patient to achieve the desired treatment, including, but not limited to the, the anterior side, the posterior side, the left side, or the right side, etc.

The elongate light-emitting catheter 230 may include a light-emitting element 232. The light-emitting element 232 may be configured to emit light and be disposed at the position adjacent to the inner wall the stent 10. In some embodiments, the light-emitting element 232 may be configured to emit “green” light having a wavelength in a range from about 495 nanometers to about 570 nanometers. In some embodiments, the light-emitting element 232 may be configured to emit light radially about at least a portion of the elongate light-emitting catheter 230. For instance, the light-emitting element 232 may be configured to emit light radially along a 360 degree arc extending about at least a portion of the elongate light-emitting catheter 230 at which the light-emitting element 232 is disposed. For instance, in some embodiments, the light-emitting element 232 may be disposed at a distal tip of the elongate light-emitting catheter 230, as illustrated in FIG. 4. In some embodiments, the light-emitting element 232 may be configured to emit light at least radially about the distal tip of the elongate light-emitting catheter 230. Having the light-emitting element 232 be configured to emit light radially promote aspects herein such as causing the photodegradable polymer in the photolabile layer to undergo a depolymerization reaction, as detailed herein. In some embodiments, the light-emitting element 232 may be a light-emitting diode (LED) element or may be fiber-optic element that is coupled to a light source that is external to the elongate light-emitting catheter 230. For instance, the light-emitting element 232 may be an internal light-emitting diode (LED) element that is coupled to the distal end (e.g., which is implanted in the patient) of the elongate light-emitting catheter 230. However, in some embodiments, the light-emitting element can be a fiber-optic element or other type of element that is coupled to an external laser or other type of light source that is external to the patient. In another embodiment, a light-emitting element (not shown) may be incorporated into the body of the stent 10 and activated non-invasively through electrical induction (wireless energy transfer). In another embodiment, the light-emitting element in the stent 10 may include materials that undergo X-ray-mediated luminescence (XML) which are embedded in the stent 10 and later stimulated to emit light upon X-ray exposure (e.g., to externally generated X-ray radiation). That is, in some embodiments, the stent 10 includes a light-emitting element configured to apply the light to the photolabile layer responsive to an external application of X-ray radiation, electromagnetic induction, or both, to the light-emitting element of the implanted stent. In such embodiments, the light-emitting element of the stent 10 may be employed alone or may be employed in conjunction with the light-emitting element 232 of the elongate light-emitting catheter 230.

As noted, application of in vivo light to a photolabile layer including a photodegradable polymer may cause the photodegradable polymer to undergo a depolymerization reaction. The application of the in vivo light can continue at least until the photolabile layer undergoes the depolymerization reaction to an extent sufficient to liquify the photodegradable polymer and thereby cause the photolabile layer to mechanically detach from an adjacent material, as described herein.

As noted above, the selective liquification of the photolabile layer may cause internal material, such as, but not limited to, sludge, bile, stone debris, etc., coupled to a surface (e.g., an inner surface) of the photolabile layer (prior to application of the in vivo light) to be dislodged or slough off from the stent 10, thus mitigating occlusion of a lumen of the stent 10. It is contemplated that if sludge, bile, stone debris, biofilm, etc., are left undisturbed they may cause early occlusion or other malfunctioning of the stent 10.

It is contemplated that the elongated tubular member of the stent can be made from a number of different materials such as, but not limited to, metals, metal alloys, shape memory alloys, and/or polymers, as desired, enabling the stent to be expanded into shape when accurately positioned within the body. In some instances, the material may be selected to enable the stent to be removed with relative ease as well. For example, the elongated tubular member of the stent can be formed from alloys such as, but not limited to, nitinol and Elgiloy®. Depending on the material selected for construction, the stent may be self-expanding or require an external force to expand the stent. In some embodiments, composite filaments may be used to make the stent, which may include, for example, an outer shell or cladding made of nitinol and a core formed of platinum or other radiopaque material. It is further contemplated the elongated tubular member of the stent may be formed from polymers including, but not limited to, polyethylene terephthalate (PET). In some instances, the filaments of the stent, or portions thereof, may be bioabsorbable or biodegradable, while in other instances the filaments of the stent, or portions thereof, may be biostable and/or bio-inert.

The stents, delivery systems, and the various components thereof, may be made from a metal, metal alloy, polymer (some examples of which are disclosed below), a metal-polymer composite, ceramics, proteins, or combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys, nickel-copper alloys, nickel-cobalt-chromium-molybdenum alloys, nickel-molybdenum alloys, other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys; platinum enriched stainless steel; titanium; combinations thereof; and the like; or any other suitable material.

Some examples of suitable polymers for the stents or delivery systems may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), polycaprolactone (PCL), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. For instance, in some embodiments, the photodegradable polymer described herein may comprises a poly(olefin sulfone), a polythiophene, a polycarbodiimide, a nitrobenzyl group containing polyamide, polyester, polyether, polycarbamate, and/or derivatives and co-polymers thereof.

Examples of suitable polysulfones may include poly(1-butene sulfone) (PBS), poly(1-pentene sulfone) (PPS), poly(1-hexene sulfone) (PHS), poly(1-octene sulfone) (POS), poly(1-cyclopentene sulfone) (PcycloPS), poly(2-methyl-1butene sulfone) (PMBS), poly 2-methyl-1-pentene sulfone) (PMPS), poly(2-methyl-1-hexene sulfone) (PMNS), and poly(cyclohexene sulfone) (PcycloHS), among others and derivatives and co-polymers thereof.

Examples of suitable nitrobenzyl group containing polymers and co-polymers including poly(nitrobenzyl methacrylate), poly(nitrobenzyl acrylate), and poly(nitrobenzyl ether), poly(nitrobenzyl carbamate), poly(nitrobenzyl amide), poly(nitrobenzyl ester), among others and derivatives and co-polymers thereof.

In at least some embodiments, portions or all of the stents or delivery systems may also be doped with, made of, or otherwise include a radiopaque material. Radiopaque materials are generally understood to be materials which are opaque to RF energy in the wavelength range spanning x-ray to gamma-ray (at thicknesses of <0.005″). These materials are capable of producing a relatively dark image on a fluoroscopy screen relative to the light image that non-radiopaque materials such as tissue produce. This relatively bright image aids the user of the stents or delivery systems in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stents or delivery systems to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A stent, the stent comprising:
an elongated tubular member forming a tubular wall, the elongated tubular member configured to move between a radially collapsed configuration and a radially expanded configuration;
a polymer coating disposed on a surface of the tubular wall; and
a photolabile layer disposed on an inner surface of the polymer coating, wherein the photolabile layer includes a photodegradable polymer.

2. The stent of claim 1, wherein the polymer coating comprises silicone or polyurethane.

3. The stent of claim 1, wherein the polymer coating extends from a proximal end to a distal end of the elongated tubular member, and wherein the photolabile layer extends from the proximal end to the distal end of the elongated tubular member.

4. The stent of claim 1, wherein the polymer coating comprises an opaque filler, a reflective filler, a remotely-stimulated light source, or any combination thereof.

5. The stent of claim 1, wherein the photodegradable polymer is configured to undergo a depolymerization reaction responsive to application of visible light.

6. The stent of claim 5, wherein the visible light has a wavelength in a range from about 495 nanometers to about 570 nanometers.

7. The stent of claim 1, wherein the photodegradable polymer is configured to undergo a depolymerization reaction responsive to application of ultraviolet light, X-ray radiation, or electromagnetic induction.

8. The stent of claim 7, wherein the ultraviolet light has a wavelength in a range from 200 nanometers to 380 nanometers.

9. The stent of claim 1, wherein the photolabile layer includes a pharmacological agent.

10. The stent of claim 9, wherein the pharmacological agent is an antiproliferative agent.

11. The stent of claim 9, wherein the pharmacological agent is an antimicrobial agent.

12. The stent of claim 1, wherein the photodegradable polymer further comprises a poly(olefin sulfone), a polythiophene, a polycarbodiimide, a nitrobenzyl group containing polyamide, polyester, polyether, polycarbamate, and/or derivatives and co-polymers thereof.

13. The stent of claim 1, wherein the photodegradable polymer comprises about 50 weight percent to 100 weight percent of a total weight of the photolabile layer.

14. The stent of claim 1, wherein the photodegradable polymer comprises about 100 weight percent of a total weight of the photolabile layer.

15. The stent of claim 1, wherein the photolabile layer comprises a plurality of photolabile layers, the plurality of photolabile layers including:
a second photolabile layer disposed on an inner surface of the polymer coating, the second photolabile layer configured to undergo depolymerization in response to an applied light having a second wavelength; and
a first photolabile layer disposed on an inner surface of the second photolabile layer, the first photolabile layer configured to undergo depolymerization in response to an applied light having a second wavelength.

16. A system comprising:
a stent including:
an elongated tubular member forming a tubular wall, the elongated tubular member configured to move between a radially collapsed configuration and a radially expanded configuration;
a polymer coating disposed on a surface of the tubular wall;
a photolabile layer disposed on an inner surface of the polymer coating, wherein the photolabile layer includes a photodegradable polymer;
and
a light-emitting element, wherein in response to emission of light by the light-emitting element at least a portion of the photolabile layer is configured to liquify and mechanically decouple from a remaining portion of the photolabile layer, the polymer coating, or both.

17. A method for restoring patency of a stent implanted in a body of a patient, the method comprising:
applying light to a photolabile layer of the implanted stent; and
wherein in response to the application of the light, at least a portion of the photolabile layer undergoes depolymerization to form a liquified portion of the photolabile layer.

18. The method of claim 17, wherein the liquified portion of the photolabile layer is configured to mechanically decouple from a remaining portion of the photolabile layer, a polymer coating of the implanted stent, or both.

19. The method of claim 17, wherein the light-emitting element is a remotely-stimulated light-emitting element included in the stent, and wherein the remotely-stimulated light-emitting element is configured to apply the light in-vivo of the body of the patient to the photolabile layer responsive to an external and/or transdermal application of X-ray radiation, electromagnetic induction, or both, to the remotely-stimulated light-emitting element.

20. The method of claim 17, wherein light-emitting element is included in a light-emitting catheter, and wherein the light-emitting element in the light emitting catheter is configured to apply the light in vivo of the body of the patient to the photolabile layer.
